(19) United States
(12) Patent Application Publication
(10) Pub. No.: US 2010/0150849 A1
(43) Pub. Date: Jun. 17, 2010

(54) Oral Care Compositions

(76) Inventors: William E. Prosise, Ramsey, NJ (US); Jay Hilsenbeck, Wayne, NJ (US)

Correspondence Address: INTERNATIONAL SPECIALTY PRODUCTS;WILLIAM J. DAVIS, ESQ., 1361 ALPS ROAD, WAYNE, NJ, 07470, US

(21) Appl. No.: 12/374,423

(22) PCT Filed: Jul. 26, 2007
(86) PCT No.: PCT/US07/74429

§ 371 Date: Dec. 3, 2009

Related U.S. Application Data
(60) Provisional application No. 60/833,498, filed on Jul. 26, 2006.

(51) Int. Cl. A61K 8/97; A61K 8/36; A61Q 11/00

(52) U.S. Cl. 424/58; 424/49

(57) ABSTRACT

An oral care composition comprising a copolymer of an alkyl vinyl ether and maleic acid or anhydride, a surface active vegetable gum, and a non-cationic antibacterial agent. The composition exhibits increased uptake and retention of the non-cationic antibacterial agent on application to teeth.

### CROSS-REFERENCE TO RELATED APPLICATIONS

The present application claims the benefit of U.S. Provisional Application Ser. No. 60/833,498, filed on Jul. 26, 2006, the disclosure of which is hereby incorporated by reference in its entirety.

### FIELD OF THE INVENTION

This invention relates to oral care compositions, such as toothpaste compositions, and more particularly to such compositions containing a copolymer of an alkylvinyl ether and maleic acid or maleic anhydride, e.g., Gantrez® S-97 (ISP), and an additive which is a surface active vegetable gum as an effective antibacterial enhancing agent (AEA) for a non-cationic antibacterial agent, e.g., triclosan. The compositions exhibit high uptake and retention of the antibacterial agent on teeth.

### BACKGROUND OF THE INVENTION

Oral care compositions comprising non-cationic antibacterial agents and copolymers of maleic acid or maleic anhydride and an alkyl vinyl ether, e.g., methylvinyl ether-maleic acid anhydride, and other polycarboxylates are well known in the art. See, for example, U.S. Patent Nos. 6,692,725; 5,776,435; 5,686,064; 5,582,816; 5,538,715; 5,531,982; 5,496,540; 5,472,685; 5,466,437; 5,453,265; 5,368,844; 5,356,615; 5,344,641; 5,334,325; 5,312,618; 5,294,431; 5,292,526; 5,288,480; 5,279,813; 5,275,805; 5,273,741; 5,260,062; 5,256,401; 5,192,531; 5,192,530; 5,188,821; 5,180,578; 5,178,851; 5,167,951; 5,156,835; 5,135,738; 5,080,887; 5,037,637; 5,032,386; and 4,894,220.

However, none of these patented compositions exhibit a particularly high uptake and retention of the antibacterial agent on teeth. It would be desirable for improved dental health to provide oral care compositions with enhanced uptake and retention of an antibacterial agent on teeth. Accordingly, it is an object of this invention to improve the efficacy of oral care compositions containing a non-cationic antibacterial agent.

### SUMMARY OF THE INVENTION

We have discovered that oral care compositions comprising a copolymer of an alkylvinyl ether and maleic acid or maleic anhydride, a surface active vegetable gum and a non-cationic antibacterial agent exhibit unexpectedly high uptake and retention of the non-cationic antibacterial agent on teeth.

In accordance with this invention, there is provided oral care compositions comprising a copolymer of an alkyl vinyl ether and maleic acid or anhydride, a surface active vegetable gum and a non-cationic antibacterial agent. The oral care compositions exhibit increased uptake and retention of the non-cationic antibacterial agent on application to teeth.

In another embodiment of the invention, there is provided a method of enhancing the uptake and/or retention of a non-cationic antibacterial agent on teeth, the method comprising delivering said non-cationic antimicrobial agent to the teeth in combination with a copolymer of maleic acid or maleic anhydride and an alkyl vinyl ether and a surface active vegetable gum.

### BRIEF DESCRIPTION OF THE FIGURES

FIG. 1 shows the procedure used for testing the efficacy of uptake/retention of the non-cationic antibacterial agent triclosan on mucin-coated HAP disks.

FIG. 2 is a graphical representation of uptake and retention of triclosan from a composition of maleic acid-methyl vinyl ether polymer and surface active vegetable gum, i.e., gum arabic (invention) vs. a control containing a maleic acid-methyl vinyl ether copolymer and no surface active vegetable gum.

### DETAILED DESCRIPTION OF THE INVENTION

While the invention is described hereinafter with respect to preferred embodiments thereof, e.g., a maleic acid or maleic anhydride alkyl vinyl ether copolymer in combination with the non-cationic antibacterial agent triclosan and the surface active vegetable gum arabic, the invention can also be practiced in connection with other non-cationic antibacterial agents such as thymol, phenol and other phenolics and with other surface active vegetable gums such as hydroxypropylmethylcellulose or karaya gum.

The oral care compositions can contain up to about 10% by weight of the maleic acid or maleic anhydride alkyl vinyl ether copolymers. In toothpastes, the maleic acid or maleic anhydride alkyl vinyl ether copolymers can be present in an amount of 0.1-5 wt %, preferably 0.5-3 wt %.

The non-cationic antibacterial agent can be present in an amount of about 0.01-5 wt %, preferably 0.1-4 wt % and more preferably 0.2-3 wt %.

The surface active vegetable gum can be present in an amount of about 0.001-5 wt %, preferably 0.01-4 wt % and more preferably 0.05-3 wt %.

The oral care composition can take the form of any conventional oral care composition, In addition to toothpastes, the oral care composition can be in the form of a denture adhesive, a mouth wash, a lozenge, chewing gum and the like.

The specific viscosity (SV) of the maleic acid or maleic anhydride alkyl vinyl ether copolymers can be in the range of 0.1 to about 10, preferably 0.1-6 and more preferably 0.2-6. As used herein, “specific viscosity” refers to the viscosity of the alkyl vinyl ether maleic anhydride copolymer in a suitable solvent divided by the viscosity of the solvent.

In a particularly preferred embodiment, the non-cationic antibacterial agent is present in the presence of a known surface active agent such as sodium lauryl sulfate. Other surface active agents normally used in oral care compositions can also be used. An example is Poloxamers. A preferred Poloxamer is Poloxamer 407 available from BASF Corp.

In yet another embodiment, this invention can be practiced in conjunction with multimodel blends of copolymers of maleic acid or maleic anhydride and an alcylvinylether as described more fully in commonly assigned PCT International Application entitled “ORAL CARE COMPOSITIONS” filed on even date herewith, the disclosure of which is hereby incorporated by reference in its entirety. For example, compositions of this invention can contain a bimodal blend of such copolymers having specific viscosities in the ranges of 2-10 and 0.1-2 respectively.

### EXAMPLE

### Toothpaste (T/P) Mixtures for Triclosan Uptake and Retention Tests

Stock T/P Mixture without abrasives:

### Add:

61.00 g Water
20.00 g 70% Sorbitol
13.00 g Propylene glycol
2.46 g Sodium lauryl sulfate
0.30 g Non-cationic antibacterial (triclosan)
1.00 g Ethanol
0.24 g NaF
1.80 g Maleic acid-vinyl ether copolymer* (Gantrez-97) *suitably present in an amount of 0.01-5% by wt in the composition.
0.20 g Gum Arabic** **0.1 to 1% by wt.

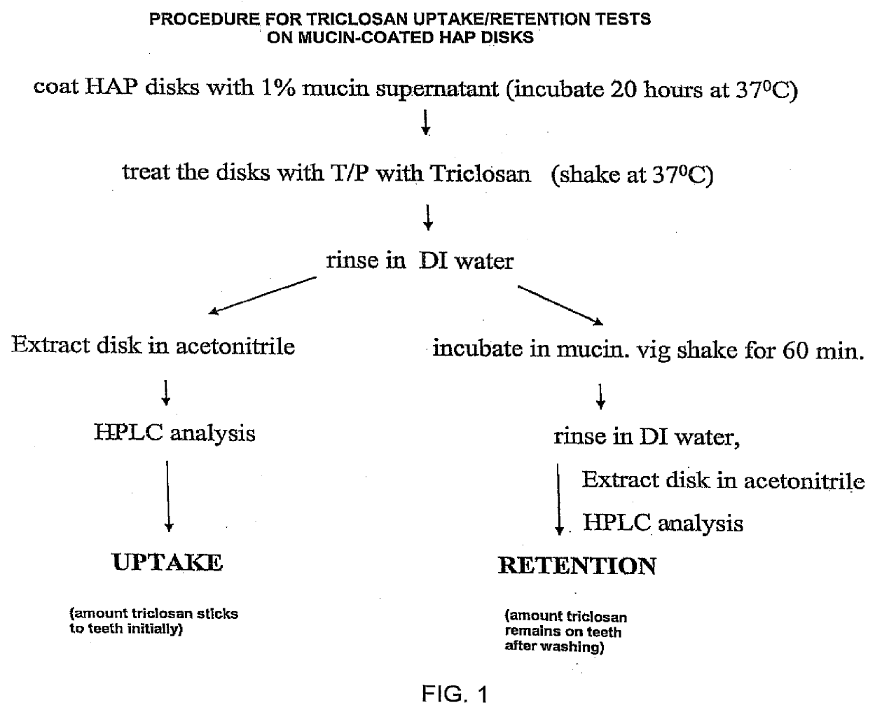

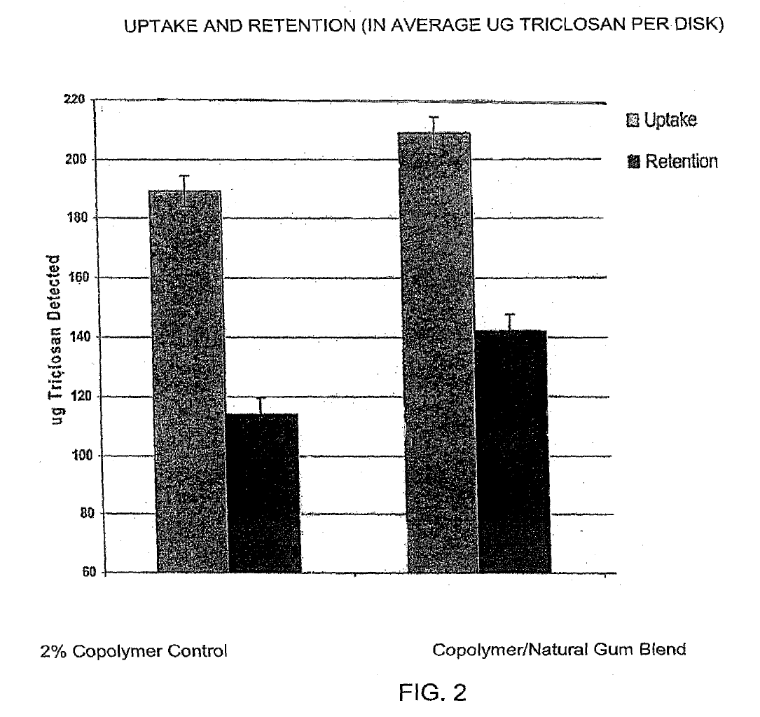

The procedure used for testing the efficacy of the compositions of the invention is shown in FIG. 1. The results of such testing are shown in FIG. 2 which demonstrate that the gum arabic containing oral care compositions of this invention exhibit increased uptake and retention of the non-cationic antibacterial agent triclosan as compared to the control.

What is claimed:

1. An oral care composition comprising:
a) a copolymer of an alkyl vinyl ether and maleic acid or anhydride
b) a surface active vegetable gum, and
c) a non-cationic antibacterial agent, wherein said composition exhibits increased uptake and retention of said antibacterial agent to teeth.

2. The oral care composition of claim 1 in wherein said alkyl vinyl ether is methyl vinyl ether.

3. The oral care composition of claim 1 wherein said non-cationic antibacterial agent is triclosan.

4. The oral care composition of claim 1 wherein said surface active vegetable gum is gum Arabic.

5. The oral care composition of claim 1 wherein said copolymer is present in an amount of 0.01-5 wt %, and said substance is an amount of about 0.1-1 wt %.

6. The oral care composition of claim 1 wherein said non-cationic antibacterial agent is present in an amount of about 0.1-1 wt %.

7. The oral care composition of claim 1 wherein said surface active vegetable gum is present in an amount of 0.01-10 wt %.

8. An oral care composition comprising a copolymer of maleic anhydride or maleic acid and a methyl vinyl ether, triclosan and gum acacia.

9. A method of enhancing the uptake and/or retention of a non-cationic antibacterial agent on teeth, said method comprising delivering said non-cationic antimicrobial agent to said teeth in combination with a copolymer of maleic acid or maleic anhydride and an alkyl vinyl ether and a surface active vegetable gum.
